(19) United States
(12) Patent Application Publication
(10) Pub. No.: US 2022/0079504 A1
(43) Pub. Date: Mar. 17, 2022

(54) BRAIN FUNCTION MEASUREMENT DEVICE

(71) Applicant: SHIMADZU CORPORATION, Kyoto-shi, Kyoto (JP)

(72) Inventors: Shumpei YAMAGUCHI, Kyoto-shi, Kyoto (JP); Akihiro ISHIKAWA, Kyoto-shi, Kyoto (JP)

(73) Assignee: SHIMADZU CORPORATION, Kyoto-shi, Kyoto (JP)

(21) Appl. No.: 17/423,319

(22) PCT Filed: Dec. 23, 2019
(86) PCT No.: PCT/JP2019/050293

§ 371 Date: Jul. 15, 2021

(30) Foreign Application Priority Data
Feb. 8, 2019 (JP) 2019-012922

(51) Int. Cl. A61B 5/00; A61B 5/026; A61B 5/11; A61B 5/0205; G16H 40/63

(52) U.S. Cl. CPC A61B 5/4064; A61B 5/026; A61B 5/1118; A61B 5/024; A61B 5/7278; G16H 40/63; A61B 5/0205

(57) ABSTRACT

A brain function measurement device (100) includes a brain blood flow information acquirer (1) configured to acquire brain blood flow information (d1) of a subject (P), a rest information acquirer (2) configured to acquire rest information (d5) to determine whether or not the subject is at rest, and a controller (3) configured to acquire, as resting measurement data (d2), the brain blood flow information with the rest information satisfying a predetermined condition based on the rest information acquired by the rest information acquirer.

### TECHNICAL FIELD

The present invention relates to a brain function measurement device, and more particularly, it relates to a brain function measurement device that measures brain function at rest.

### BACKGROUND ART

Conventionally, a brain function measurement device that measures brain function at rest is known. Such a brain function measurement device is disclosed in Japanese Patent Laid-Open No. 2015-116213, for example.

A brain function measurement device disclosed in Japanese Patent Laid-Open No. 2015-116213 measures the subject's brain blood flow at rest, and calculates the functional connection (correlation) of brain regions between a plurality of predetermined regions based on the measured brain blood flow.

### PRIOR ART

### Patent Document

### Patent Document 1: Japanese Patent Laid-Open No. 2015-116213

### SUMMARY OF THE INVENTION

### Problems to be Solved by the Invention

When the subject's brain blood flow is measured, the brain may not be in a resting state even when the subject is at rest at first glance. In other words, in general, being at rest refers to a state in which a person is not in a motional state, such as sitting or lying down, and is not given an active task or external stimulus, and an apparent resting state is different from a resting state of brain activity. Even when the subject's body is in a resting state, the brain may not be in a resting state when the subject is thinking about something, for example. The resting state of the brain indicates that the subject does not feel unpleasant external stimuli such as stress, and does not intentionally think about anything.

However, it is conceivable that the brain function measurement device disclosed in Japanese Patent Laid-Open No. 2015-116213 does not determine whether or not the subject's brain is in a resting state when measuring the subject's brain blood flow at rest. Therefore, when the functional connection of the brain regions between the plurality of predetermined regions obtained from the measurement results is analyzed, the measurement result in a case in which the brain is not in a resting state may be included while the reliability of the measurement results may be reduced, and the accuracy of analysis of resting brain function may be reduced.

The present invention is intended to solve at least one of the above problems. The present invention aims to provide a brain function measurement device that enables improvement of the reliability of the measurement results and improvement of the accuracy of analysis of resting brain function.

### Means for Solving the Problems

In order to attain the aforementioned object, a brain function measurement device according to a first aspect of the present invention includes a brain blood flow information acquirer configured to acquire brain blood flow information of a subject, a rest information acquirer configured to acquire rest information to determine whether or not the subject is at rest, and a controller configured to acquire, as resting measurement data, the brain blood flow information with the rest information satisfying a predetermined condition based on the rest information acquired by the rest information acquirer.

As described above, the brain function measurement device according to the first aspect of the present invention includes the controller configured to acquire, as the resting measurement data, the brain blood flow information with the rest information satisfying the predetermined condition to determine whether or not the subject is at rest. Accordingly, acquisition of the brain blood flow information in a case in which the brain of the subject is not at rest as the resting measurement data can be significantly reduced or prevented. Therefore, the reliability of the measurement results can be improved, and thus analysis using the measurement results with the improved reliability becomes possible. Consequently, the accuracy of analysis of resting brain function can be improved.

In the aforementioned brain function measurement device according to the first aspect, the controller is preferably configured to generate cumulative resting measurement data obtained by accumulating the resting measurement data based on the resting measurement data. Accordingly, brain function can be analyzed with the cumulative resting measurement data generated from the resting measurement data. Consequently, brain function can be efficiently analyzed as compared with a case in which brain function is analyzed using data in which a resting state and a non-resting state are mixed.

In this case, the controller is preferably configured to acquire one or a plurality of pieces of the resting measurement data and combine the resting measurement data in order of acquisition to generate one piece of the cumulative resting measurement data. Accordingly, brain function can be analyzed using the cumulative resting measurement data obtained by combining the resting measurement data in time series. Consequently, one or a plurality of pieces of resting measurement data are combined in time series, and thus only the resting measurement data can be analyzed over time such that brain function can be more accurately and easily analyzed.

In the aforementioned configuration in which the cumulative resting measurement data is generated, the brain blood flow information acquirer is preferably configured to continuously acquire the brain blood flow information during acquisition of the rest information, and the controller is preferably configured to extract information in a resting state as the resting measurement data from the continuously acquired brain blood flow information, and generate the cumulative resting measurement data based on the extracted resting measurement data. Accordingly, the cumulative resting measurement data can be generated by extracting the resting measurement data from the brain blood flow information while concurrently acquiring the rest information and the brain blood flow information. Consequently, as compared with a case in which only the brain blood flow information in a resting state is acquired as the resting measurement data by switching whether or not the brain blood flow information is acquired according to the resting state and non-resting state of the brain, the resting measurement data can be extracted from the continuously acquired brain blood flow information without switching whether or not the brain blood flow information is acquired, and thus complex control of the measurement process can be significantly reduced or prevented.

In this case, the controller is preferably configured to perform, on the continuously acquired brain blood flow information, a process to enable distinction between the resting state and a non-resting state based on the rest information. Accordingly, data from the start to the end of a brain function test of the subject can be acquired. Consequently, brain function can be analyzed using the measurement results of the entire brain function test, and thus brain function can be easily analyzed. Furthermore, it is possible to distinguish between the resting state and the non-resting state of the brain in the continuously acquired brain blood flow information, and thus the knowledge such as the distribution of the resting state and the non-resting state of the brain in the brain blood flow information can be obtained.

In the aforementioned configuration in which the resting measurement data is extracted from the continuously acquired brain blood flow information to generate the cumulative resting measurement data, the controller is preferably configured to acquire the brain blood flow information as the resting measurement data when the rest information satisfies the predetermined condition for a predetermined time or longer. Accordingly, even when the brain is in a resting state, acquisition as the resting measurement data can be significantly reduced or prevented when the resting state of the brain does not continue for the predetermined time or longer. Consequently, when the resting state of the brain is shortened, e.g., the resting state and the non-resting state are alternately repeated, such that the resting measurement data cannot be treated as significant data, extraction of the resting measurement data for generating the cumulative resting measurement data can be significantly reduced or prevented. Therefore, the reliability of the cumulative resting measurement data can be further improved, and thus the accuracy of analysis of brain function can be further improved.

In the aforementioned configuration in which the resting measurement data is extracted from the continuously acquired brain blood flow information to generate the cumulative resting measurement data, the controller is preferably configured to terminate a measurement process when a cumulative measurement time of the resting measurement data reaches a predetermined measurement time. Accordingly, the measurement process can be terminated based on the cumulative measurement time of the resting measurement data. Consequently, for example, as compared with a case in which an operator terminates the measurement process when the cumulative measurement time of the resting measurement data reaches a predetermined time, usability (convenience for the operator) can be improved.

In the aforementioned brain function measurement device according to the first aspect, the rest information acquirer is preferably configured to acquire heartbeat information of the subject as the rest information, and the controller is preferably configured to acquire parasympathetic nerve activity based on a fluctuation in a heartbeat time interval of the subject and to determine a resting state of the subject's brain. Accordingly, the parasympathetic nerve activity is acquired based on the fluctuation in the heartbeat time interval such that the resting state of the brain can be determined. Consequently, the resting state of the brain can be determined by acquiring the heartbeat information, and thus it is possible to easily determine whether or not the brain is in a resting state.

In this case, the controller is preferably configured to perform power spectrum analysis on the fluctuation in the heartbeat time interval of the subject to acquire an HF component, which is an indicator of the parasympathetic nerve activity, and determine a state in which an intensity of the HF component exceeds a predetermined intensity as the resting state of the brain. Accordingly, the resting state of the brain can be determined based on the HF component, which is an indicator of the parasympathetic nerve activity. Consequently, it is possible to determine the resting state of the brain by confirming whether or not the intensity of the HF component exceeds the predetermined intensity, and thus it is possible to more easily determine whether or not the brain is in a resting state.

A brain function measurement device according to a second aspect of the present invention includes a brain blood flow information acquirer configured to acquire brain blood flow information of a subject, a rest information acquirer configured to acquire rest information to determine whether or not the subject is at rest, and a controller configured to start acquiring the brain blood flow information as resting measurement data when the subject is at rest based on the rest information acquired by the rest information acquirer, and to stop acquiring the brain blood flow information when the subject is no longer at rest.

As described above, the brain function measurement device according to the second aspect of the present invention includes the controller configured to start acquiring the brain blood flow information as the resting measurement data when the subject is at rest based on the rest information acquired by the rest information acquirer, and to stop acquiring the brain blood flow information when the subject is no longer at rest. Accordingly, similarly to the aforementioned brain function measurement device according to the first aspect, the accuracy of analysis of resting brain function can be improved. Furthermore, the brain blood flow information is acquired only when the subject is at rest, and thus unlike a configuration in which the brain blood flow information at rest is acquired as the resting measurement data from the brain blood flow information acquired in a state in which both rest and non-rest are mixed, the brain blood flow information can be acquired as the resting measurement data without determining whether or not the subject is at rest after the brain blood flow information is acquired.

### Effect of the Invention

According to the present invention, as described above, it is possible to provide the brain function measurement device that enables improvement of the reliability of the measurement results and improvement of the accuracy of analysis of brain function at rest.

### BRIEF DESCRIPTION OF THE DRAWINGS

FIG. 1 is a schematic view showing the overall configuration of a brain function measurement device according to a first embodiment of the present invention.

FIG. 2 is a schematic view showing the configuration of a brain blood flow information acquirer according to the first embodiment of the present invention.

FIG. 3A is a schematic view illustrating brain blood flow data and resting measurement data, and FIG. 3B is a schematic view illustrating cumulative resting measurement data.

FIGS. 4A to 4C are schematic views illustrating a process for a controller to determine the resting state of the subject's brain according to the first embodiment of the present invention.

FIG. 5 is a flowchart illustrating a process for the brain function measurement device to generate the cumulative resting measurement data according to the first embodiment of the present invention.

FIG. 6 is a schematic view illustrating a process to enable a controller to distinguish brain blood flow information according to a second embodiment of the present invention.

FIG. 7 is a flowchart illustrating a process for a brain function measurement device to generate cumulative resting measurement data according to the second embodiment of the present invention.

FIG. 8 is a schematic view illustrating a process to enable a controller to distinguish brain blood flow information according to a third modified example of the present invention.

FIG. 9 is a flowchart illustrating a process for a brain function measurement device to generate cumulative resting measurement data according to the third modified example of the present invention.

FIG. 10 is a schematic view showing the overall configuration of a brain function measurement device according to a first modified example of the present invention.

FIG. 11 is a schematic view showing the overall configuration of a brain function measurement device according to a second modified example of the present invention.

### MODES FOR CARRYING OUT THE INVENTION

Embodiments embodying the present invention are hereinafter described on the basis of the drawings.

### First Embodiment

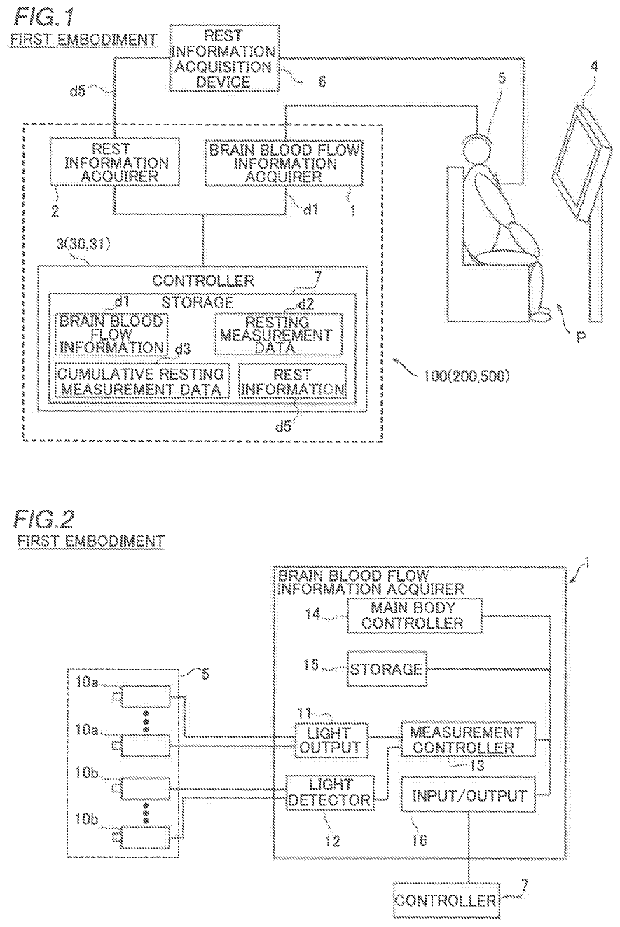

The configuration of a brain function measurement device 100 according to a first embodiment of the present invention is now described with reference to FIGS. 1 to 5.

### Configuration of Brain Function Measurement Device

First, the configuration of the brain function measurement device 100 according to the first embodiment of the present invention is described with reference to FIG. 1.

As shown in FIG. 1, the brain function measurement device 100 includes a brain blood flow information acquirer 1, a rest information acquirer 2, and a controller 3.

The brain function measurement device 100 is a device (optical measurement device) that optically measures the brain activity of a subject P using near-infrared spectroscopy (NIRS) and generates time-series measurement result data, for example.

In the first embodiment, the brain function measurement device 100 measures the brain activity of the subject P when the subject P is performing a task displayed on a display 4. The subject P is tasked with continuing to see marks such as + and ⊚ displayed on the display 4, for example. The display 4 includes a liquid crystal monitor, for example.

The brain blood flow information acquirer 1 acquires brain blood flow information d1 of the subject P based on an input signal from the controller 3. The detailed configuration of the brain blood flow information acquirer 1 is described below.

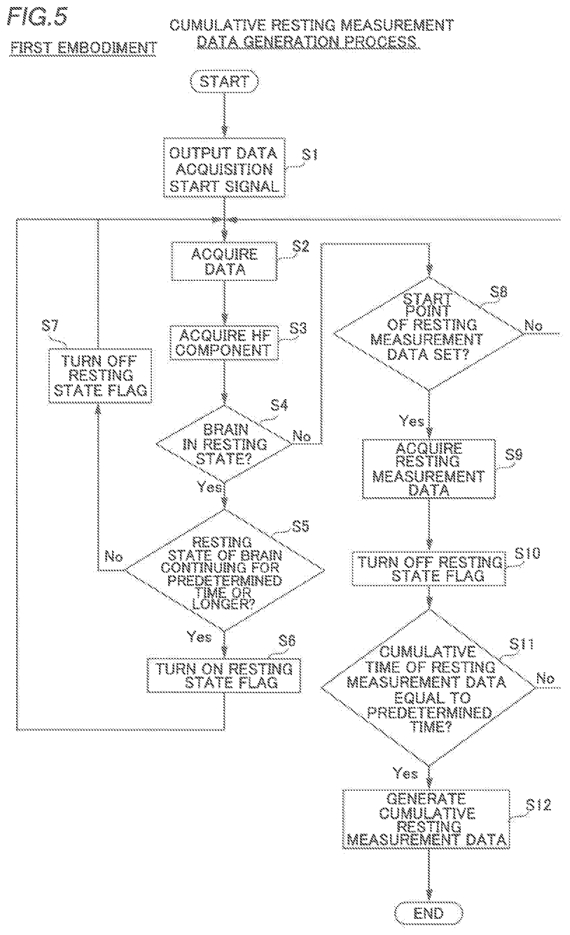

The rest information acquirer 2 acquires rest information d5 to determine whether or not the subject P is at rest. Specifically, the rest information acquirer 2 acquires heartbeat information d5a (see FIG. 4A) of the subject P as the rest information d5. The rest information acquirer 2 is configured as a so-called input/output interface.

A rest information acquisition device 6 acquires the rest information d5 of the subject P based on an input signal from the controller 3. Specifically, the rest information acquisition device 6 acquires the heartbeat information d5a of the subject P. The rest information acquisition device 6 includes an electrocardiogram device, for example. The rest information acquisition device 6 includes a plurality of electrodes attached to the subject P, a controller that generates an electrocardiogram based on the potential of the heart of the subject P acquired by the plurality of electrodes, a display that displays the electrocardiogram generated by the controller, etc.

The controller 3 acquires brain blood flow information d1 in which the brain of the subject P is in a resting state among pieces of brain blood flow information d1 as resting measurement data d2. Furthermore, the controller 3 generates cumulative resting measurement data d3 obtained by accumulating the resting measurement data d2 based on the resting measurement data d2. Moreover, the controller 3 outputs, to each of the brain blood flow information acquirer 1 and the rest information acquisition device 6, a signal to synchronize measurements in the brain blood flow information acquirer 1 and the rest information acquisition device 6. The controller 3 includes a central processing unit (CPU), for example. A detailed configuration in which the controller 3 acquires the resting measurement data d2 and a detailed configuration in which the controller 3 generates the cumulative resting measurement data d3 are described below.

The controller 3 includes a storage 7. The storage 7 stores the brain blood flow information d1, the rest information d5, the resting measurement data d2, the cumulative resting measurement data d3, etc. The storage 7 includes a hard disk drive (HDD) or a non-volatile memory, for example.

### Configuration of Brain Blood Flow Information Acquirer

Next, the configuration of the brain blood flow information acquirer 1 is described with reference to FIG. 2.

The brain blood flow information acquirer 1 acquires the brain blood flow information d1 of the subject P using measurement probes (light transmitting probes 10a and light receiving probes 10b) connected thereto via optical fibers.

As shown in FIG. 2, the brain blood flow information acquirer 1 includes the light transmitting probes 10a, the light receiving probes 10b, a light output 11, a light detector 12, a measurement controller 13, a main body controller 14, a storage 15, and an input/output 16.

The light transmitting probes 10a and the light receiving probes 10b of the brain blood flow information acquirer 1 are respectively placed at predetermined positions on the head surface of the subject P by being attached to a probe fixing holder 5 attached to the head of the subject P.

The light output 11 is configured to output, to the light transmitting probes 10a, a plurality of measurement lights in a near-infrared wavelength region. The light output 11 includes a semiconductor laser, for example. The light detector 12 acquires and detects measurement light incident on the light receiving probes 10b via the optical fibers. The light detector 12 includes a photomultiplier tube, for example.

The measurement controller 13 measures brain function with the measurement probes (the light transmitting probes 10a and the light receiving probes 10b) placed on the head of the subject P. The main body controller 14 is a computer including a CPU, a memory, etc., and functions as the main body controller 14 of the brain blood flow information acquirer 1 by executing various programs stored in the storage 15. The storage 15 includes an HDD, for example, and is configured to store control programs executed by the main body controller 14 and setting information and to store the brain blood flow information d1 obtained as a result of measurement. The input/output 16 is an interface for connecting to an external device in the brain function measurement device 100, for example.

In the first embodiment, the light output 11 emits measurement light in the near-infrared wavelength region from the light transmitting probes 10a placed on the head surface of the subject P. Then, the light detector 12 acquires the intensity of the measurement light (the amount of received light) by allowing the measurement light reflected in the head to enter the light receiving probes 10b placed on the head surface and detecting the measurement light. A plurality of light transmitting probes 10a and a plurality of light receiving probes 10b are provided, and are attached to the holder 5 configured to fix each probe at a predetermined position on the head surface. The measurement controller 13 measures the amount of change in oxygenated hemoglobin, the amount of change in deoxygenated hemoglobin, and the amount of change in total hemoglobin based on the intensity of the measurement light (the amount of received light) at a plurality of wavelengths (three wavelengths of 780 nm, 805 nm, and 830 nm, for example) and the absorption characteristics of hemoglobin. The measurement controller 13 acquires the brain blood flow information d1 of the subject P by acquiring the amount of change in hemoglobin. The brain blood flow information acquirer 1 outputs the acquired brain blood flow information d1 to the controller 3 of the brain function measurement device 100 via the input/output 16.

### Acquisition of Resting Measurement Data d2 and Generation of Cumulative Resting Measurement Data

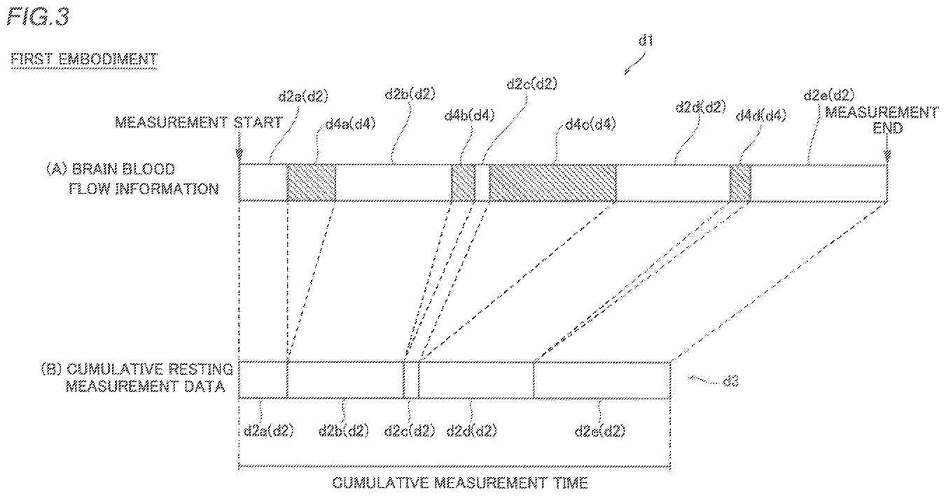

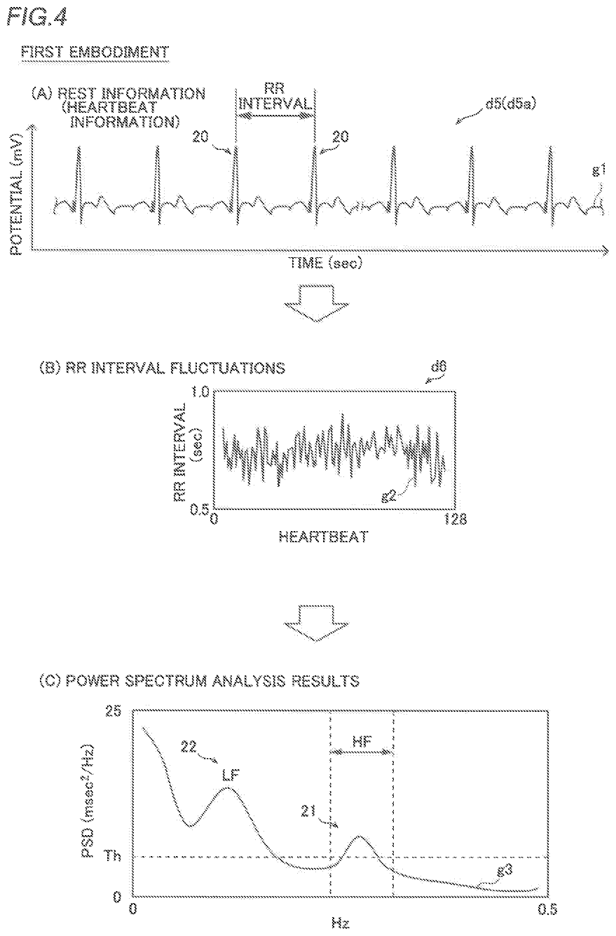

Next, a configuration in which the controller 3 acquires the resting measurement data d2 and a configuration in which the controller 3 generates the cumulative resting measurement data d3 are described with reference to FIG. 3.

FIG. 3A is a schematic view showing the brain blood flow information d1 of the subject P. The brain blood flow information d1 shown in FIG. 3A includes the resting measurement data d2 in which the subject P's brain is at rest and non-resting measurement data d4 in which the subject P's brain is not at rest (in a non-resting state). In FIG. 3A, diagonal hatching is added to the non-resting brain blood flow information d1 (non-resting measurement data d4) for convenience.

Unlike a physical resting state, the resting state of the brain cannot necessarily be freely controlled by the subject P. Therefore, the resting state of the brain does not necessarily continue for the desired measurement time, and may occur intermittently as shown in FIG. 3A. Therefore, the controller 3 is configured to acquire, as the resting measurement data d2, the brain blood flow information d1 with the rest information d5 satisfying a predetermined condition based on the rest information d5 acquired by the rest information acquirer 2. Specifically, the controller 3 is configured to acquire, as the resting measurement data d2, the brain blood flow information d1 in which the brain of the subject P is determined to be in a resting state among the pieces of brain blood flow information d1. A method for the controller 3 to determine the resting state of the brain is described below.

In an example shown in FIG. 3, the controller 3 is configured to acquire resting measurement data d2a, resting measurement data d2b, resting measurement data d2c, resting measurement data d2d, and resting measurement data d2e from the brain blood flow information d1.

In the first embodiment, the controller 3 acquires a plurality of pieces of resting measurement data d2 and combines the plurality of pieces of resting measurement data d2 in the order of acquisition to generate one piece of cumulative resting measurement data d3. Specifically, the brain blood flow information acquirer 1 continuously acquires the brain blood flow information d1 while acquiring the rest information d5, and the controller 3 extracts information in a resting state as the resting measurement data d2 from the continuously acquired brain blood flow information d1, and generates the cumulative resting measurement data d3 based on the extracted resting measurement data d2.

In the example shown in FIG. 3, the controller 3 combines the acquired plurality of pieces of resting measurement data d2 (resting measurement data d2a to resting measurement data d2e) in the order of acquisition to generate one piece of cumulative resting measurement data d3. Therefore, the cumulative resting measurement data d3 includes only the resting measurement data d2 obtained by removing the non-resting measurement data d4 (d4a to d4d) from the brain blood flow information d1.

In the first embodiment, the controller 3 acquires the brain blood flow information d1 as the resting measurement data d2 when the rest information d5 satisfies the predetermined condition for a predetermined time or longer. The controller 3 terminates a measurement process when the cumulative measurement time of the resting measurement data d2 reaches a predetermined measurement time. In the first embodiment, the cumulative measurement time is set to eight minutes, for example. The measurement time (time stamp) of each resting measurement data d2 is not continuous, and thus the controller 3 reassigns the time stamps in the order of combination when generating the cumulative resting measurement data d3.

In the first embodiment, the controller 3 stores the acquired brain blood flow information d1, rest information d5, and resting measurement data d2 and the generated cumulative resting measurement data d3 in the storage 7. The brain blood flow information d1 includes the resting measurement data d2 and the non-resting measurement data d4, and thus the brain blood flow information d1 may be data for a time longer than the predetermined measurement time.

### Determination of Resting State

Next, a configuration in which the controller 3 according to the first embodiment determines whether or not the brain of the subject P is in a resting state is described with reference to FIG. 4.

FIG. 4A is a schematic view of a graph g1 showing the rest information d5 of the subject P acquired by the rest information acquisition device 6. The horizontal axis of the graph g1 represents time (seconds), and the vertical axis thereof represents potential (mV). FIG. 4B is a schematic view of a graph g2 showing fluctuations in an RR interval of the rest information d5 (fluctuations d6 in a heartbeat time interval). The horizontal axis of the graph g2 represents the number of beats (corresponding to a time axis), and the vertical axis thereof represents the RR interval. The RR interval refers to an interval between adjacent R waves 20 in the graph g1. FIG. 4C is a schematic view of a graph g3 showing the results of power spectrum analysis of the graph g2 showing the fluctuations in the RR interval of the rest information d5. The horizontal axis of the graph g3 represents frequency, and the vertical axis thereof represents power spectral density (PSD) (msec²/Hz).

In the first embodiment, the controller 3 acquires the fluctuation d6 in the heartbeat time interval of the subject P based on the heartbeat information d5a of the subject P acquired as the rest information d5. Specifically, the controller 3 takes a predetermined number of heartbeats of the subject P as one group and acquires the fluctuations d6 in the heartbeat time interval based on the RR interval of the heartbeat information d5a in one group. The predetermined number of heartbeats included in one group is 128, for example.

In the first embodiment, the controller 3 acquires the parasympathetic nerve activity based on the fluctuations d6 in the heartbeat time interval of the subject P and determines the resting state of the brain of the subject P. Specifically, as shown in FIG. 4B, the controller 3 acquires the graph g2 showing the fluctuations in the RR interval as indicators of the fluctuations d6 in the heartbeat time interval from the heartbeat information d5a (rest information d5) of the subject P. Then, the controller 3 acquires the graph g3 by performing power spectrum analysis on the graph g2. The power spectrum analysis indicates acquiring a frequency spectrum showing the intensity of a frequency component included in the fluctuations d6 in the heartbeat time interval by Fourier transforming the fluctuations d6 in the heartbeat time interval.

Two peaks are formed in the power spectral density (graph g3) of the RR interval. Among frequency components in which these two peaks are formed, the high frequency peak is referred to as an HF component 21, and the low frequency peak is referred to as a LF component 22. The HF component 21 reflects the parasympathetic nerve activity, and the LF component 22 reflects the sympathetic nerve activity. When the parasympathetic nerve is dominant, the brain is considered to be in a relaxed and resting state. Therefore, in this description, the resting state of the brain is defined as a state in which the intensity of the HF component 21 exceeds a predetermined threshold in the power spectral density (graph g3).

Specifically, the controller 3 acquires the HF component 21, which is an indicator of the parasympathetic nerve activity, by performing power spectrum analysis on the fluctuations d6 (graph g2) in the heartbeat time interval of the subject P, and determines a state in which the intensity of the HF component 21 exceeds a predetermined intensity Th as the resting state of the brain. That is, a case in which the rest information d5 satisfies the predetermined condition refers to a state in which the intensity of the HF component 21 exceeds the predetermined intensity Th. The HF component 21 refers to a frequency component included in a band having a frequency of about 0.20 to about 0.40 Hz in the graph g3.

The controller 3 is configured to generate the graph g3 by shifting a range of acquiring the number of heartbeats from the heartbeat information d5a to data measured after each point. Therefore, the controller 3 can determine the resting state of the subject P's brain over time.

### Cumulative Resting Measurement Data Generation Process

Next, a series of process operations for the controller 3 according to the first embodiment to generate the cumulative resting measurement data d3 is described with reference to FIG. 5.

In step S1, the controller 3 outputs a data acquisition start signal to the brain blood flow information acquirer 1 and the rest information acquisition device 6 based on an input operation of an operator. Then, in step S2, the controller 3 acquires the brain blood flow information d1 and the rest information d5. Then, the process advances to step S3.

In step S3, the controller 3 acquires the HF component 21 based on the rest information d5. Then, in step S4, the controller 3 determines whether or not the brain of the subject P is in a resting state. That is, the controller 3 determines whether or not the brain of the subject P is in a resting state depending on whether or not the intensity of the HF component 21 exceeds the predetermined intensity Th. When the brain of the subject P is in a resting state, the process advances to step S5. When the brain of the subject P is in a non-resting state, the process advances to step S8.

In step S5, the controller 3 determines whether or not the resting state of the brain of the subject P has continued for the predetermined time or longer. When the resting state of the brain of the subject P has continued for the predetermined time or longer, the process advances to step S6. When the resting state of the brain of the subject P has not continued for the predetermined time or longer, the process advances to step S7.

In step S6, the controller 3 turns on a resting state flag indicating that the brain of the subject P is in a resting state. Specifically, when determining that the brain of the subject P is in a resting state, the controller 3 stores a value (1, for example) indicating the resting state in the storage 7. Then, the process returns to step S2.

In step S7, the controller 3 turns off the resting state flag. Specifically, when determining that the brain of the subject P is in a non-resting state, the controller 3 overwrites the value (1, for example) indicating the resting state stored in the storage 7 with a value (0, for example) indicating the non-resting state. When the resting state flag is not turned on, the process returns to step S2.

In step S8, the controller 3 determines whether or not the start point of the resting measurement data d2 is set in the brain blood flow information d1. When the start point of the resting measurement data d2 is set in the brain blood flow information d1, the process advances to step S9. When the start point of the resting measurement data d2 is not set in the brain blood flow information d1, the process returns to step S2.

In step S9, the controller 3 acquires the resting measurement data d2. Specifically, the controller 3 acquires, as the resting measurement data d2, the brain blood flow information d1 during a period in which the brain of the subject P continues to be in a resting state. Then, in step S10, the controller 3 turns off the resting state flag. Then, the process advances to step S11.

Then, in step S11, the controller 3 determines whether or not the cumulative measurement time of the resting measurement data d2 exceeds the predetermined time. When the cumulative measurement time of the resting measurement data d2 exceeds the predetermined time, the process advances to step S12. When the cumulative measurement time of the resting measurement data d2 does not exceed the predetermined time, the process returns to step S9.

In step S12, the controller 3 generates one piece of cumulative resting measurement data d3 from a plurality of pieces of resting measurement data d2, and terminates the process.

### Advantages of First Embodiment

In the first embodiment, the following advantages are obtained.

In the first embodiment, as described above, the brain function measurement device 100 includes the brain blood flow information acquirer 1 configured to acquire the brain blood flow information d1 of the subject P, the rest information acquirer 2 configured to acquire the rest information d5 to determine whether or not the subject P is at rest, and the controller 3 configured to acquire, as the resting measurement data d2, the brain blood flow information d1 with the rest information d5 satisfying the predetermined condition based on the rest information d5 acquired by the rest information acquirer 2. Accordingly, acquisition of the brain blood flow information d1 in a case in which the brain of the subject P is not at rest as the resting measurement data d2 can be significantly reduced or prevented. Therefore, the reliability of the measurement results can be improved, and thus analysis using the measurement results with the improved reliability becomes possible. Consequently, the accuracy of analysis of resting brain function can be improved.

In the first embodiment, as described above, the controller 3 is configured to generate the cumulative resting measurement data d3 obtained by accumulating the resting measurement data d2 based on the resting measurement data d2. Accordingly, brain function can be analyzed with the cumulative resting measurement data d3 generated from the resting measurement data d2. Consequently, brain function can be efficiently analyzed as compared with a case in which brain function is analyzed using data in which a resting state and a non-resting state are mixed.

In the first embodiment, as described above, a plurality of pieces of resting measurement data d2 are acquired and combined in the order of acquisition to generate one piece of cumulative resting measurement data d3. Accordingly, brain function can be analyzed using the cumulative resting measurement data d3 obtained by combining the resting measurement data d2 in time series. Consequently, a plurality of pieces of resting measurement data d2 are combined in time series, and thus only the resting measurement data d2 can be analyzed over time such that brain function can be more accurately and easily analyzed.

In the first embodiment, as described above, the brain blood flow information acquirer 1 is configured to continuously acquire the brain blood flow information d1 during acquisition of the rest information d5, and the controller 3 is configured to extract the information in a resting state as the resting measurement data d2 from the continuously acquired brain blood flow information d1, and generates the cumulative resting measurement data d3 based on the extracted resting measurement data d2. Accordingly, the cumulative resting measurement data d3 can be generated by extracting the resting measurement data d2 from the brain blood flow information d1 while concurrently acquiring the rest information d5 and the brain blood flow information d1. Consequently, as compared with a case in which only the brain blood flow information d1 in a resting state is acquired as the resting measurement data d2 by switching whether or not the brain blood flow information d1 is acquired according to the resting state and non-resting state of the brain, the resting measurement data d2 can be extracted from the continuously acquired brain blood flow information d1 without switching whether or not the brain blood flow information d1 is acquired, and thus complex control of the measurement process can be significantly reduced or prevented.

In the first embodiment, as described above, the controller 3 is configured to acquire the brain blood flow information d1 as the resting measurement data d2 when the rest information d5 satisfies the predetermined condition for the predetermined time or longer. Accordingly, even when the brain is in a resting state, acquisition as the resting measurement data d2 can be significantly reduced or prevented when the resting state of the brain does not continue for the predetermined time or longer. Consequently, when the resting state of the brain is shortened, e.g., the resting state and the non-resting state are alternately repeated, such that the resting measurement data d2 cannot be treated as significant data, extraction of the resting measurement data d2 for generating the cumulative resting measurement data d3 can be significantly reduced or prevented. Therefore, the reliability of the cumulative resting measurement data d3 can be further improved, and thus the accuracy of analysis of brain function can be further improved.

In the first embodiment, as described above, the controller 3 is configured to terminate the measurement process when the cumulative measurement time of the resting measurement data d2 reaches the predetermined measurement time. Accordingly, the measurement process can be terminated based on the cumulative measurement time of the resting measurement data d2. Consequently, for example, as compared with a case in which the operator terminates the measurement process when the cumulative measurement time of the resting measurement data d2 reaches a predetermined time, usability (convenience for the operator) can be improved.

In the first embodiment, as described above, the rest information acquirer 2 is configured to acquire the heartbeat information d5a of the subject P as the rest information d5, and the controller 3 is configured to acquire the parasympathetic nerve activity based on the fluctuations d6 in the heartbeat time interval of the subject P and to determine the resting state of the brain of the subject P. Accordingly, the parasympathetic nerve activity is acquired based on the fluctuations d6 in the heartbeat time interval such that the resting state of the brain can be determined. Consequently, the resting state of the brain can be determined by acquiring the heartbeat information d5a, and thus it is possible to easily determine whether or not the brain is in a resting state.

In the first embodiment, as described above, power spectrum analysis is performed on the fluctuations d6 in the heartbeat time interval of the subject P such that the HF component 21, which is an indicator of the parasympathetic nerve activity, is acquired, and a state in which the intensity of the HF component 21 exceeds the predetermined intensity Th is determined as the resting state of the brain. Accordingly, the resting state of the brain can be determined based on the HF component 21, which is an indicator of the parasympathetic nerve activity. Consequently, it is possible to determine the resting state of the brain by confirming whether or not the intensity of the HF component 21 exceeds the predetermined intensity Th, and thus it is possible to more easily determine whether or not the brain is in a resting state.

### Second Embodiment

A brain function measurement device 200 according to a second embodiment of the present invention is now described with reference to FIGS. 1, 6, and 7. In the second embodiment, a controller 30 is configured to perform, on continuously acquired brain blood flow information d1, a process to enable distinction between a resting state and a non-resting state based on rest information d5, unlike the first embodiment in which the resting measurement data d2 is extracted from the brain blood flow information d1 and the cumulative resting measurement data d3 is generated. The same or similar configurations as those of the first embodiment are denoted by the same reference numerals, and description thereof is omitted.

In the second embodiment in which acquisition of the brain blood flow information d1 and acquisition of the rest information d5 are performed concurrently, the controller 30 performs, on the continuously acquired brain blood flow information d1, the process to enable distinction between a resting state and a non-resting state based on the rest information d5.

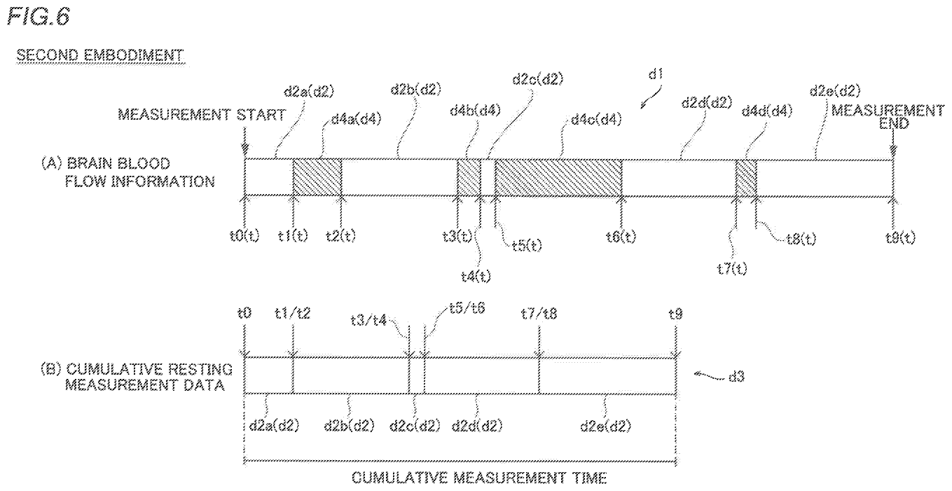

FIG. 6A is a schematic view showing an example in which the process to enable distinction between a resting state and a non-resting state is performed on the continuously acquired brain blood flow information d1.

As shown in FIG. 6A, in the second embodiment, the controller 30 performs the process to enable distinction between a resting state and a non-resting state in the brain blood flow information d1 by assigning a time stamp t (time stamps t0 to t9) to the brain blood flow information d1 when the brain of a subject P is in a resting state. In the example shown in FIG. 6A, the time stamps t0, t2, t4, t6, and t8 indicate the start points of resting measurement data d2. Furthermore, in the example shown in FIG. 6A, the time stamps t1, t3, t5, t7, and t9 indicate the end points of the resting measurement data d2.

That is, in the example shown in FIG. 6A, the brain blood flow information d1 between the time stamp t0 and the time stamp t1, the brain blood flow information d1 between the time stamp t2 and the time stamp t3, the brain blood flow information d1 between the time stamp t4 and the time stamp t5, the brain blood flow information d1 between the time stamp t6 and the time stamp t7, and the brain blood flow information d1 between the time stamp t8 and the time stamp t9 are the resting measurement data d2 (resting measurement data d2a to d2e, respectively).

In the second embodiment, the controller 30 displays the brain blood flow information d1 that has undergone the process to enable distinction between a resting state and a non-resting state on a display (not shown). Therefore, an operator such as a doctor or an engineer can obtain knowledge such as the distribution of a resting state in the brain blood flow information d1 by checking the brain blood flow information d1 displayed on the display. The display includes a liquid crystal monitor, for example.

### Cumulative Resting Measurement Data Generation Process

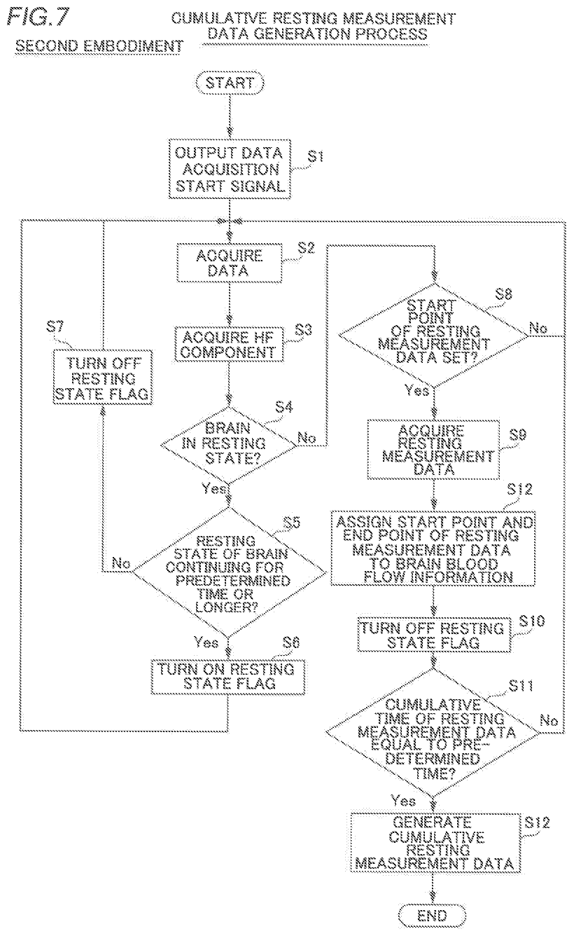

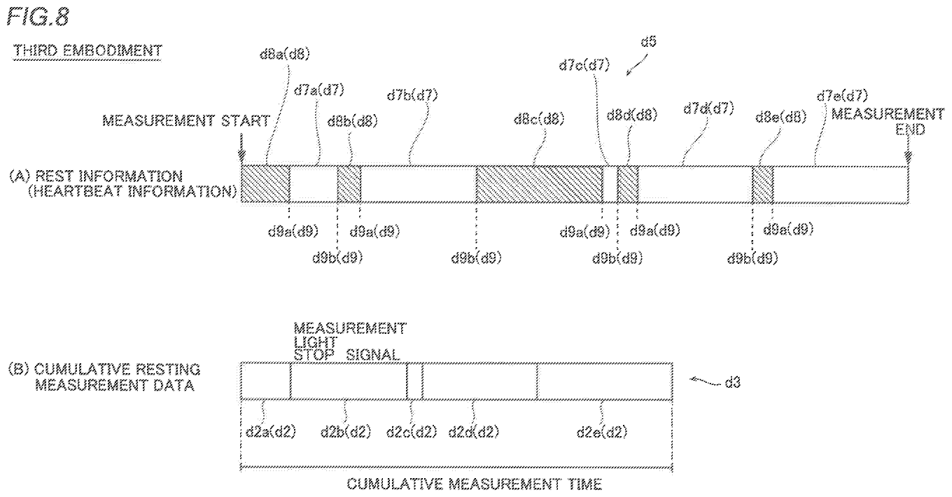

A series of process operations for the controller 30 according to the second embodiment to generate cumulative resting measurement data d3 is now described with reference to FIG. 7. The process operations in step S1 to step S12 are similar to those of the first embodiment, and thus detailed description thereof is omitted.

In step S1 to step S9, the controller 30 acquires the resting measurement data d2. Then, the process advances to step S12.

In step S12, the controller 30 assigns the time stamps t (time stamps t0 to t9) to the continuously acquired brain blood flow information d1 at portions corresponding to the resting measurement data d2. Then, the process advances from step S10 to step S12, and the controller 30 generates the cumulative resting measurement data d3 and terminates the process.

The remaining configurations of the second embodiment are similar to those of the first embodiment.

### Advantages of Second Embodiment

In the second embodiment, the following advantages are obtained.

In the second embodiment, as described above, the controller 30 is configured to perform, on the continuously acquired brain blood flow information d1, the process to enable distinction between a resting state and a non-resting state based on the rest information d5. Accordingly, data from the start to the end of a brain function test of the subject P can be acquired. Consequently, brain function can be analyzed using the measurement results of the entire brain function test, and thus brain function can be easily analyzed. Furthermore, it is possible to distinguish between the resting state and the non-resting state of the brain in the continuously acquired brain blood flow information d1, and thus the knowledge such as the distribution of the resting state and the non-resting state of the brain in the brain blood flow information d1 can be obtained.

The remaining advantages of the second embodiment are similar to those of the first embodiment.

### Third Embodiment

A brain function measurement device 500 (see FIG. 1) according to a third embodiment of the present invention is now described with reference to FIGS. 1, 8, and 9. In the third embodiment, a controller 31 (see FIG. 1) is configured to start acquiring brain blood flow information d1 as resting measurement data d2 when a subject P is at rest based on rest information d5 acquired by a rest information acquirer 2, and to stop acquiring the brain blood flow information d1 when the subject P is no longer at rest, unlike the first embodiment in which the resting measurement data d2 is extracted from the brain blood flow information d1 and the cumulative resting measurement data d3 is generated. The same or similar configurations as those of the first embodiment are denoted by the same reference numerals, and description thereof is omitted.

As shown in FIG. 1, the brain function measurement device 500 includes a brain blood flow information acquirer 1 that acquires the brain blood flow information d1 of the subject P, the rest information acquirer 2 that acquires the rest information d5 to determine whether or not the subject P is at rest, and the controller 31 that starts acquiring the brain blood flow information d1 as the resting measurement data d2 when the subject P is at rest based on the rest information d5 acquired by the rest information acquirer 2, and stops acquiring the brain blood flow information d1 when the subject P is no longer at rest.

As shown in FIG. 8A, in the third embodiment, the controller 31 acquires the rest information d5 of the subject P. The controller 31 determines whether or not the brain of the subject P is in a resting state based on the rest information d5. A configuration in which the controller 31 determines whether or not the brain of the subject P is in a resting state is similar to that of the controller 3 in the first embodiment, and thus detailed description thereof is omitted.

In the third embodiment, the controller 31 outputs a brain blood flow information acquisition control signal d9 to the brain blood flow information acquirer 1 based on the resting state of the brain of the subject P. Specifically, the controller 31 outputs a brain blood flow information acquisition start signal d9a to the brain blood flow information acquirer 1 when the brain of the subject P is in a resting state. Furthermore, the controller 31 outputs a brain blood flow information acquisition stop signal d9b to the brain blood flow information acquirer 1 when the brain of the subject P is not in a resting state.

That is, in an example shown in FIG. 8A, a period from the output of the brain blood flow information acquisition start signal d9a to the output of the brain blood flow information acquisition stop signal d9b is a rest period d7 (d7a to d7e). Furthermore, a period from the output of the brain blood flow information acquisition stop signal d9b to the output of the brain blood flow information acquisition start signal d9a is a non-rest period d8 (d8a to d8e). The controller 31 acquires the brain blood flow information d1 during the rest period d7 (d7a to d7e) as the resting measurement data d2 (d2a to d2e), and generates cumulative resting measurement data d3.

### Cumulative Resting Measurement Data Generation Process

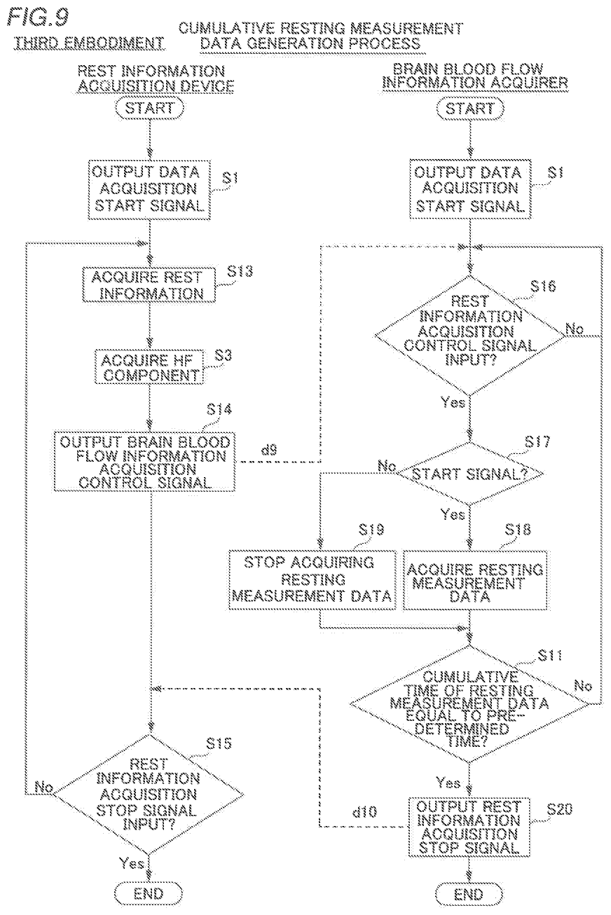

A series of process operations for the controller 31 according to the third embodiment to generate the cumulative resting measurement data d3 is now described with reference to FIG. 9. The process operations in step S1, step S3, and step S11 are similar to those of the first embodiment, and thus detailed description thereof is omitted. In the following, for convenience, the process operations by a rest information acquisition device 6 and the process operations by the brain blood flow information acquirer 1 are described individually, but in reality, these process operations are performed concurrently.

### Process Operations by Rest Information Acquisition Device

In step S1, the controller 31 outputs a data acquisition start signal to the rest information acquisition device 6 based on an input operation of an operator. Next, in step S13, the controller 31 acquires the rest information d5. Then, the process advances to step S3.

In step S3, the controller 3 acquires an HF component 21 based on the rest information d5. Then, in step S14, the controller 31 determines the resting state of the brain of the subject P based on the HF component 21, and outputs the brain blood flow information acquisition control signal d9 (the brain blood flow information acquisition start signal d9a or the brain blood flow information acquisition stop signal d9b) to the brain blood flow information acquirer 1.

Next, in step S15, the controller 31 determines whether or not a rest information acquisition stop signal d10 has been input. When the rest information acquisition stop signal d10 has been input, the process terminates. When the rest information acquisition stop signal d10 has not been input, the process advances to step S13.

### Process Operations by Brain Blood Flow Information Acquirer

In step S1, the controller 31 outputs a data acquisition start signal to the brain blood flow information acquirer 1 based on the input operation of the operator. The process operation in step S1 in the process of the rest information acquisition device 6 and the process operation in step S1 in the process of the brain blood flow information acquirer 1 are performed in synchronization with each other.

Next, in step S16, the controller 31 determines whether or not the brain blood flow information acquisition control signal d9 has been input. When the brain blood flow information acquisition control signal d9 has been input, the process advances to step S17. When the brain blood flow information acquisition control signal d9 has not been input, the controller 31 repeats the process operation in step S16.

In step S17, the controller 31 determines whether the brain blood flow information acquisition control signal d9 is the brain blood flow information acquisition start signal d9a or the brain blood flow information acquisition stop signal d9b. When the brain blood flow information acquisition control signal d9 is the brain blood flow information acquisition start signal d9a, the process advances to step S18. When the brain blood flow information acquisition control signal d9 is the brain blood flow information acquisition stop signal d9b, the process advances to step S19.

When the process advances to step S18, in step S18, the brain blood flow information acquirer 1 starts acquiring the brain blood flow information d1 as the resting measurement data d2. When the brain blood flow information acquirer 1 has started acquiring the brain blood flow information d1 when the process advances to step S18, the process operation in step S18 is omitted. When the process advances to step S19, in step S19, the brain blood flow information acquirer 1 stops acquiring the brain blood flow information d1. When the brain blood flow information acquirer 1 has not started acquiring the brain blood flow information d1 when the process advances to step S19, the process operation in step S19 is omitted.

Next, in step S11, the controller 31 determines whether or not the cumulative measurement time of the resting measurement data d2 exceeds a predetermined time. When the cumulative measurement time of the resting measurement data d2 exceeds the predetermined time, the process advances to step S20. When the cumulative measurement time of the resting measurement data d2 does not exceed the predetermined time, the process advances to step S16.

In step S20, the controller 31 outputs the rest information acquisition stop signal d10 to the rest information acquisition device 6, and terminates the process.

The remaining configurations of the third embodiment are similar to those of the first embodiment.

### Advantages of Third Embodiment

In the third embodiment, the following advantages are obtained.

In the third embodiment, as described above, the brain function measurement device 500 includes the brain blood flow information acquirer 1 configured to acquire the brain blood flow information d1 of the subject P, the rest information acquirer 2 configured to acquire the rest information d5 to determine whether or not the subject P is at rest, and the controller 31 configured to start acquiring the brain blood flow information d1 as the resting measurement data d2 when the subject P is at rest based on the rest information d5 acquired by the rest information acquirer 2, and to stop acquiring the brain blood flow information d1 when the subject P is no longer at rest. Accordingly, similarly to the brain function measurement device 100 in the first embodiment, the accuracy of analysis of resting brain function can be improved. Furthermore, the brain blood flow information d1 is acquired only when the subject P is at rest, and thus unlike a configuration in which the brain blood flow information d1 at rest is acquired as the resting measurement data d2 from the brain blood flow information d1 acquired in a state in which both rest and non-rest are mixed, the brain blood flow information d1 can be acquired as the resting measurement data d2 without determining whether or not the subject P is at rest after the brain blood flow information d1 is acquired.

The remaining advantages of the third embodiment are similar to those of the first embodiment.

### Modified Examples

The embodiments disclosed this time must be considered as illustrative in all points and not restrictive. The scope of the present invention is not shown by the above description of the embodiments but by the scope of claims for patent, and all modifications (modified examples) within the meaning and scope equivalent to the scope of claims for patent are further included.

For example, while the example in which the controller 3 (30, 31) is configured to acquire the heartbeat information d5a of the subject P as the rest information d5 has been shown in each of the aforementioned first to third embodiments, the present invention is not limited to this. For example, the controller 3 (30, 31) may be configured to acquire a change in sweating of the subject P or a change in respiratory rate, for example, as the rest information d5. The controller 3 (30) may be configured to acquire any information as long as the same enables determination of whether or not the subject P's brain is at rest.

While the example in which the controller 3 (30, 31) is configured to generate the cumulative resting measurement data d3 when the cumulative measurement time of the resting measurement data d2 exceeds eight minutes has been shown in each of the aforementioned first to third embodiments, the present invention is not limited to this. The cumulative measurement time of the resting measurement data d2 may be any time length.

While the example in which the controller 3 (30) is configured to generate one piece of cumulative resting measurement data d3 from a plurality of pieces of resting measurement data d2 has been shown in each of the aforementioned first and second embodiments, the present invention is not limited to this. For example, the controller 3 (30) may be configured to generate the cumulative resting measurement data d3 from one piece of resting measurement data d2 when the measurement time of one piece of resting measurement data d2 exceeds a predetermined cumulative measurement time.

While the example in which the controller 3 (30) is configured to generate the cumulative resting measurement data d3 has been shown in each of the aforementioned first and second embodiments, the present invention is not limited to this. The controller 3 (30) may be configured not to generate the cumulative resting measurement data d3. In that case, it is only necessary to generate the brain blood flow information d1 shown in FIG. 6A.

While the example in which the controller 3 (30) is configured to combine the resting measurement data d2 in the order of acquisition to generate the cumulative resting measurement data d3 has been shown in each of the aforementioned first and second embodiments, the present invention is not limited to this. For example, the controller 3 (30) may not combine the resting measurement data d2 in the order of acquisition. However, the cumulative resting measurement data d3 becomes time-series data by combining the resting measurement data d2 in the order of acquisition, and thus it is preferable to combine the resting measurement data d2 in the order of acquisition.

While the example in which the controller 3 (30) is configured to generate the cumulative resting measurement data d3 when the cumulative measurement time of the resting measurement data d2 exceeds the predetermined time has been shown in each of the aforementioned first and second embodiments, the present invention is not limited to this. For example, the controller 3 (30) may be configured to generate the cumulative resting measurement data d3 at an arbitrary cumulative measurement time based on an input operation of the operator.

While the example in which the controller 3 (30) is configured to determine that the brain of the subject P is in a resting state when the intensity of the HF component 21 exceeds the predetermined intensity Th has been shown in each of the aforementioned first and second embodiments, the present invention is not limited to this. For example, the resting state of the brain of the subject P may be determined based on a ratio of the LF component 22 (see FIG. 4), which is a frequency component of about 0.04 to about 0.15 Hz in the graph g3 obtained by performing power spectrum analysis on the heartbeat information d5a of the subject P, and the HF component 21.

While the example in which the controller 30 is configured to perform the process to enable distinction between a resting state and a non-resting state by assigning a label 40 (labels 40a to 40e) to the brain blood flow information d1 has been shown in the aforementioned second embodiment, the present invention is not limited to this. For example, the color of the resting measurement data d2 and the color of the non-resting measurement data d4 may be different from each other such that a resting state and a non-resting state can be distinguished. Any process may be performed as long as a resting state and a non-resting state can be distinguished.

While the example in which the controller 3 (30) is configured to acquire the fluctuations d6 in the heartbeat time interval with 128 points of the heartbeat of the subject P as one group has been shown in each of the aforementioned first and second embodiments, the present invention is not limited to this. The number of heartbeats to be included in one group when the fluctuations d6 in the heartbeat time interval are acquired may be arbitrarily set.

While the example in which the brain function measurement device 100 (200) is configured to acquire the brain blood flow information d1 using NIRS has been shown in each of the aforementioned first and second embodiments, the present invention is not limited to this. For example, the brain function measurement device 100 (200) may be configured to acquire the brain blood flow information d1 by functional magnetic resonance imaging (fMRI) or single photon emission complemented tomography (SPECT), for example.

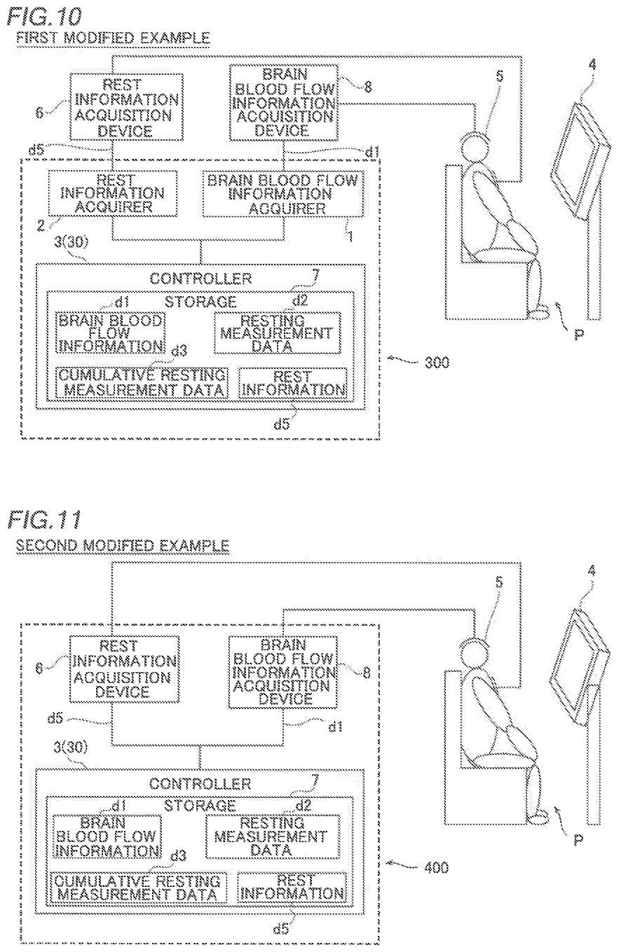

While the example in which the brain blood flow information acquirer 1 is a brain blood flow information acquisition device that acquires the brain blood flow information d1 has been shown in each of the aforementioned first and second embodiments, the present invention is not limited to this. For example, as shown in FIG. 10, a brain function measurement system 300 including a brain function measurement device 100 (200), a brain blood flow information acquisition device 8, and a rest information acquisition device 6 may be configured. When the brain function measurement system 300 is configured, a brain blood flow information acquirer 1 may be configured as an input/output interface that acquires brain blood flow information d1 acquired by the brain blood flow information acquisition device 8.

While the example in which the brain blood flow information acquirer 1 is a brain blood flow information acquisition device 8, and the rest information acquirer 2 is an input/output interface has been shown in each of the aforementioned first and second embodiments, the present invention is not limited to this. For example, as shown in FIG. 11, a brain function measurement device 400 may include a brain blood flow information acquisition device 8, a rest information acquisition device 6, and a controller 3. When the brain function measurement device 400 includes the brain blood flow information acquisition device 8 and the rest information acquisition device 6, the controller 3 may acquire brain blood flow information d1 and rest information d5 from the brain blood flow information acquisition device 8 and the rest information acquisition device 6.

While the example in which the controller 3 (31) is configured to acquire the HF component based on the fluctuations in the heartbeat time interval of the subject P and determine the resting state of the subject P, and to acquire the resting measurement data d2 based on the resting state of the subject P has been shown in each of the aforementioned first and second embodiments, the present invention is not limited to this. Regardless of the resting state of the subject P, the brain blood flow information d1 may be acquired until the resting measurement data d2 for a predetermined period of time is acquired.

While the example in which the controller 3 (30) is configured to acquire the resting measurement data d2 concurrently with acquisition of the brain blood flow information d1 and to generate the cumulative resting measurement data d3 has been shown in each of the aforementioned first and second embodiments, the present invention is not limited to this. For example, the controller 3 (30) may be configured to acquire the resting measurement data d2 after acquiring the brain blood flow information d1 and to generate the cumulative resting measurement data d3.

### DESCRIPTION OF REFERENCE NUMERALS

1: brain blood flow information acquirer
2: rest information acquirer
3, 30, 31: controller
21: HL component
100, 200: brain function measurement device
d1: brain blood flow information
d2: resting measurement data
d3: cumulative resting measurement data
d5: rest information
d5a: heartbeat information
d6: fluctuations in the heartbeat time interval
P: subject
Th: predetermined intensity

1. A brain function measurement device comprising:
a brain blood flow information acquirer configured to acquire brain blood flow information of a subject;
a rest information acquirer configured to acquire rest information to determine whether or not the subject is at rest; and
a controller configured to acquire, as resting measurement data, the brain blood flow information with the rest information satisfying a predetermined condition based on the rest information acquired by the rest information acquirer.

2. The brain function measurement device according to claim 1, wherein the controller is configured to generate cumulative resting measurement data obtained by accumulating the resting measurement data based on the resting measurement data.

3. The brain function measurement device according to claim 2, wherein the controller is configured to acquire one or a plurality of pieces of the resting measurement data and combine the resting measurement data in order of acquisition to generate one piece of the cumulative resting measurement data.

4. The brain function measurement device according to claim 2, wherein
the brain blood flow information acquirer is configured to continuously acquire the brain blood flow information during acquisition of the rest information; and
the controller is configured to:
extract information in a resting state as the resting measurement data from the continuously acquired brain blood flow information; and
generate the cumulative resting measurement data based on the extracted resting measurement data.

5. The brain function measurement device according to claim 4, wherein the controller is configured to perform, on the continuously acquired brain blood flow information, a process to enable distinction between the resting state and a non-resting state based on the rest information.

6. The brain function measurement device according to claim 4, wherein the controller is configured to acquire the brain blood flow information as the resting measurement data when the rest information satisfies the predetermined condition for a predetermined time or longer.

7. The brain function measurement device according to claim 4, wherein the controller is configured to terminate a measurement process when a cumulative measurement time of the resting measurement data reaches a predetermined measurement time.

8. The brain function measurement device according to claim 1, wherein
the rest information acquirer is configured to acquire heartbeat information of the subject as the rest information; and
the controller is configured to acquire parasympathetic nerve activity based on a fluctuation in a heartbeat time interval of the subject and to determine a resting state of the subject's brain.

9. The brain function measurement device according to claim 8, wherein
the controller is configured to:
perform power spectrum analysis on the fluctuation in the heartbeat time interval of the subject to acquire an HF component, which is an indicator of the parasympathetic nerve activity; and
determine a state in which an intensity of the HF component exceeds a predetermined intensity as the resting state of the brain.

10. A brain function measurement device comprising:
a brain blood flow information acquirer configured to acquire brain blood flow information of a subject;
a rest information acquirer configured to acquire rest information to determine whether or not the subject is at rest; and
a controller configured to start acquiring the brain blood flow information as resting measurement data when the subject is at rest based on the rest information acquired by the rest information acquirer, and to stop acquiring the brain blood flow information when the subject is no longer at rest.
